(19) United States
(12) Patent Application Publication
(10) Pub. No.: US 2015/0272506 A1
(43) Pub. Date: Oct. 1, 2015

(54) WAVE EQUATION PROCESSING

(71) Applicant: WESTERNGECO LLC, Houston, TX (US)

(72) Inventors: Paul N. Childs, Cambridge (GB); Ivan Graham, Bath (GB); James Douglas Shanks, Bath (GB); Vladimir L. Druskin, Cambridge, MA (US); Leonid Knizhzerman, Cambridge, MA (US)

(21) Appl. No.: 14/434,799

(22) PCT Filed: Oct. 9, 2013
(86) PCT No.: PCT/IB2013/059241

§ 371 Date: Apr. 10, 2015

Related U.S. Application Data
(60) Provisional application No. 61/711,999, filed on Oct. 10, 2012.

(51) Int. Cl. A61B 5/00; A61B 19/00; G01V 3/38; G01V 1/28; G01V 3/12

(52) U.S. Cl. CPC A61B 5/72; G01V 1/28; G01V 3/12; G01V 3/38; A61B 19/52; A61B 2576/00

(57) ABSTRACT

A volume of data is partitioned into a plurality of domains. A multilevel preconditioner is applied in the plurality of domains to provide iterative approximate solutions for the domains, which are coupled with boundary conditions. The approximate solutions for the domains are stitched together to provide a solution for a wave equation which can be used in full waveform inversion.

### CROSS-REFERENCE TO RELATED APPLICATION

This application claims the benefit under 35 U.S.C. §119(e) of Provisional Application No. 61/711,999, filed Oct. 10, 2012, which is hereby incorporated by reference.

### BACKGROUND

Survey data acquired for a subsurface structure can be processed to characterize the subsurface structure. The characterization of the subsurface structure can be used for various purposes, including developing an image of the subsurface structure, creating a model of the subsurface structure and/or for other purposes. By characterizing the subsurface structure, an analyst can determine if there exists an element of interest in the subsurface structure.

### SUMMARY

In general, according to some implementations, a volume of data is partitioned into a plurality of domains. A preconditioner is applied in the plurality of domains to provide iterative approximate solutions for the domains. The approximate solutions for the domains are stitched together to provide a solution for a wave equation. The wave equation solutions may be used to determine properties of and/or produce images of a subsurface structure and/or elements of the subsurface structure.

Other or additional features will become apparent from the following description, from the drawings, and from the claims.

### BRIEF DESCRIPTION OF THE DRAWINGS

Some embodiments are described with respect to the following figures.

FIG. 1 is a schematic diagram of an example arrangement for performing a survey of a subsurface structure.

FIG. 2 is a schematic diagram of partitioning a data volume into a plurality of domains, in accordance with some implementations.

FIG. 3 is a schematic diagram of decomposing a domain in a plane, or a collection of planes, according to some implementations.

FIG. 4 is a schematic diagram of stitching domains according to some implementations.

FIG. 5 is a schematic diagram of bi-frontal sweeping of domains, according to some implementations.

FIG. 6 is a block diagram of an example computer system according to some implementations.

FIG. 7 is a schematic diagram of adding a perfectly matched layer (PML) to a domain, according to further implementations.

FIG. 8 is a schematic diagram of stitching together domains using PML layers, according to some implementations.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiment(s) only, and is not intended to limit the scope, applicability or configuration of the invention. Rather, the ensuing description of the preferred exemplary embodiment(s) will provide those skilled in the art with an enabling description for implementing a preferred exemplary embodiment of the invention. It being understood that various changes may be made in the function and arrangement of elements without departing from the spirit and scope of the invention as set forth in the appended claims.

Specific details are given in the following description to provide a thorough understanding of the embodiments. However, it will be understood by one of ordinary skill in the art that the embodiments maybe practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Also, it is noted that the embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Moreover, as disclosed herein, the term “storage medium” may represent one or more devices for storing data, including read only memory (ROM), random access memory (RAM), magnetic RAM, core memory, magnetic disk storage mediums, optical storage mediums, flash memory devices and/or other machine readable mediums for storing information. The term “computer-readable medium” includes, but is not limited to portable or fixed storage devices, optical storage devices, wireless channels and various other mediums capable of storing, containing or carrying instruction(s) and/or data.

Furthermore, embodiments may be implemented by hardware, software, firmware, middleware, microcode, hardware description languages, or any combination thereof. When implemented in software, firmware, middleware or microcode, the program code or code segments to perform the necessary tasks may be stored in a machine readable medium such as storage medium that may be configured to contain non-transient instructions or the like. A processor(s) may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

Survey data of a target structure can be acquired using survey equipment. In examples where the target structure is a subsurface structure, the survey equipment can include land-based survey equipment or marine survey equipment. The survey equipment can include at least one survey source and survey receivers.

Measurement data acquired by the survey receivers is processed to characterize the subsurface structure. For example, inversion can be performed on the acquired measurement data, such as a full waveform inversion (FWI) technique or other type of inversion technique. FWI is an iterative technique for building a model (e.g. a velocity model) of the subsurface structure. Performing FWI involves solving a wave equation that provides a description of the waves that travel through various media, such as the subsurface structure, air, water, and so forth. In some examples, performing FWI may involve solving both forward and backward wave equations for a relatively large number of source points (points corresponding to locations of one or more survey sources).

In other examples, solving a wave equation can be performed as part of other processing techniques.

In the ensuing discussion, reference is made to performing processing of survey data acquired for a subsurface structure. In other implementations, processing can be performed for survey data acquired for other types of target structures, such as human or animal tissue, mechanical structures, and so forth.

Solving a wave equation in a three-dimension (3D) space can be computationally intensive, particularly when there is a relatively large amount of data to process. A wave equation can be solved either in the time domain or in the frequency domain. Techniques for solving a wave equation may be scalable. Scalability refers to the ability to partition the solving of a wave equation across multiple processing nodes to reduce elapsed computational time according to the number of processing nodes, where a “processing node” can refer to a processor, a collection of processors, a computer, a collection of computers, and so forth. The ability to partition the solving of a wave equation across multiple processing nodes allows the solving to be performed more rapidly.

Solving a wave equation in the frequency domain is less scalable. As a result, solving a wave equation in the frequency domain may not be computational feasible when the problem size or frequency is relatively large.

In accordance with some implementations, a parallel preconditioner is provided to solve a wave equation. The solving of the wave equation can be in the frequency domain. Although reference is made in the ensuing discussion to solving a wave equation in the frequency domain, it is noted that techniques or mechanisms according to some implementations can also be used to solve a wave equation in the time domain. The parallel preconditioner can partition survey data into multiple domains, such that a wave equation can be solved in the multiple domains in parallel. A preconditioner refers to an approximate solver, which provides an approximate solution of a wave equation in a respective domain.

In some implementations, the wave equation that is solved is an elastic wave equation. In other examples, the wave equation that is solved is an acoustic or pseudo-acoustic wave equation or an electromagnetic (EM) wave equation. As discussed further below, solving an acoustic wave equation may be computationally less intensive than solving an elastic wave equation.

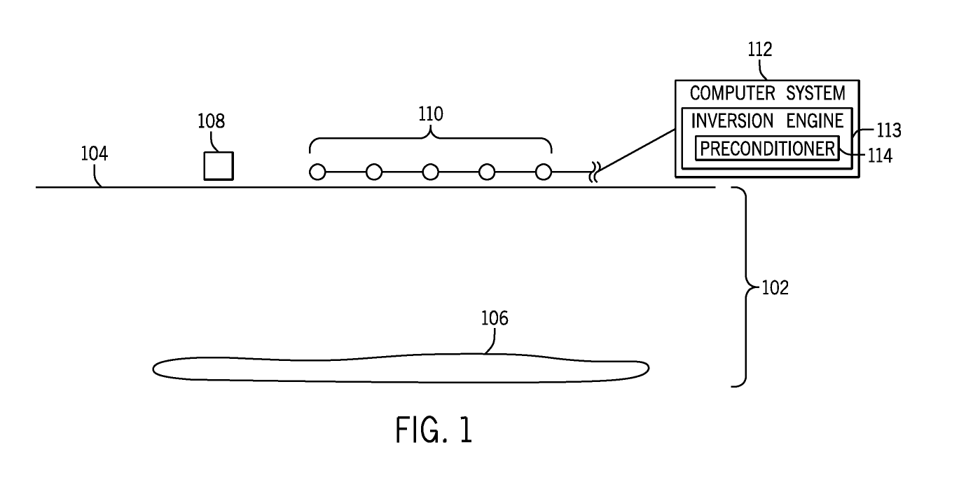

FIG. 1 illustrates an example survey equipment for surveying a subsurface structure 102 that is underneath a land surface 104. The subsurface structure 102 can include one or more elements of interest 106, such as a hydrocarbon reservoir, a freshwater aquifer, or other element of interest. The survey equipment includes at least one survey source 108 and an arrangement of survey receivers 110.

The survey source 108 can be activated to produce a survey wave that is provided into the subterranean structure 102. The survey receivers 110 can measure a response of the subterranean structure 102 to the survey wave. The collected measurement data is provided from the survey receivers 110 to a recording station, which can be part of a computer system 112. Alternatively, the recording station can be separate from the computer system 112.

The computer system 112 can perform processing of the measurement data for characterizing the subsurface structure 102. In some implementations, the computer system 112 includes an inversion engine 113 that can apply inversion (e.g. FWI) to the measurement data. The inversion engine 113 includes a preconditioner 114 according to some implementations, such as a parallel preconditioner to solve a wave equation in parallel for multiple domains.

In the example of FIG. 1, the survey equipment is a land-based survey equipment, in which the survey receivers 110 are provided on the land surface 104. In other examples, the survey equipment can be a marine survey equipment, which can include a streamer towed through a body of water, or alternatively, a seabed cable or nodes provided on the sea bottom.

In some examples, the survey equipment can include seismic survey equipment. The survey source 108 of the seismic equipment can include a seismic source, which produces seismic waves that are propagated into the subsurface structure 102. The seismic waves are reflected from the subsurface structure 102 and are measured by seismic receivers 110.

In other examples, the survey equipment can include electromagnetic (EM) survey equipment. The survey source 108 of the EM survey equipment includes an EM source that produces EM waves that are provided into the subsurface structure 102. EM waves affected by the subsurface structure 102 are measured by EM receivers 110.

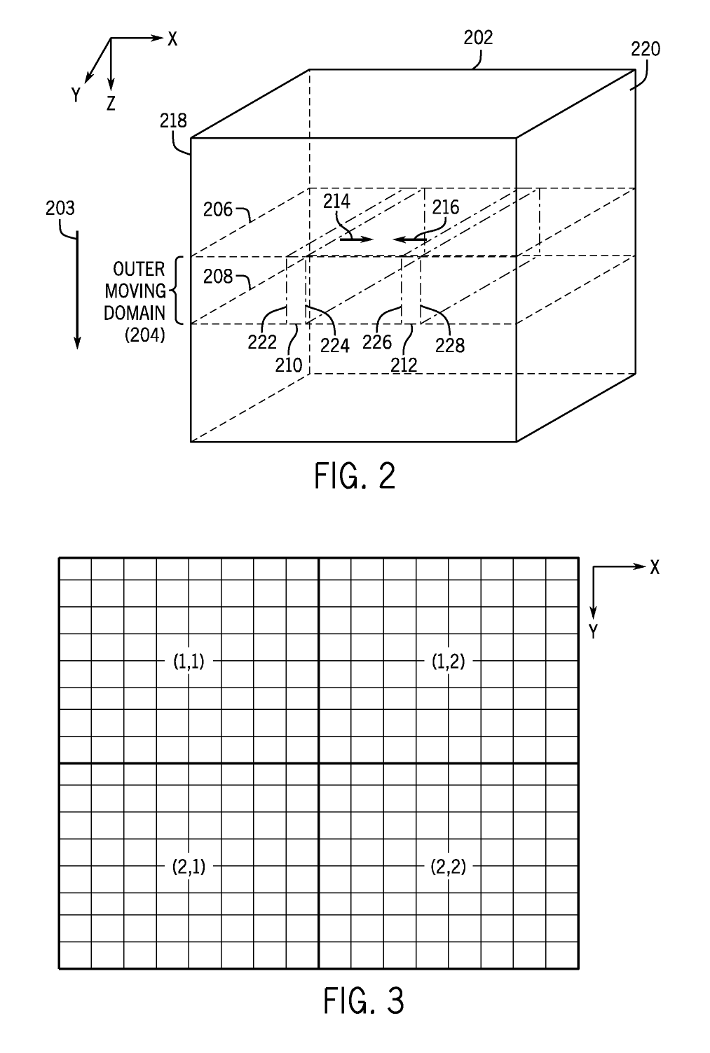

FIG. 2 illustrates the partitioning of a volume of data 202 into multiple domains. The volume of data 202 can include survey data, such as survey data acquired by the survey equipment shown in FIG. 1. Note that some preprocessing of the measured survey data may have been performed prior to provision of the volume of data 202.

To partition the volume 202, a sweep is performed in a direction 203 along the Z axis. The sweep along the direction 203 defines multiple (possibly) overlapping domains along the Z axis. A particular instance of domain 204 along the Z axis is defined between horizontal planes 206 and 208. The horizontal planes 206 and 208 are moved together along the direction 203 as part of the sweep. The spacing between the horizontal planes 206 and 208 is maintained approximately constant as the horizontal planes 206 and 208 are swept along the direction 203.

The sweep along the direction 203 is referred to as an outer sweep, and each domain 204 produced as a result of the sweep can be referred to as an outer moving domain.

Within each outer moving domain 204, further partitioning can be performed to provide inner moving domains 210 and 212 that are included within the outer moving domain 204. The inner moving domains 210 and 212 are produced by performing a sweep in respective directions 214 and 216 along the X axis. The sweep relating to the inner moving domain 210 starts at the left boundary 218 of the volume 202, while the sweep relating to the inner moving domain 212 starts at the right boundary 220.

The inner moving domain 210 is defined between a left vertical plane 222 and a right vertical plane 224. Similarly, the inner moving domain 212 is defined between a left vertical plane 226 and a right vertical plane 228. During the respective inner sweeps along the directions 214 and 216, the spacing between the vertical planes 222 and 224 is maintained approximately constant, as is the separation between vertical planes 226 and 228. The vertical planes 222, 224 are swept along direction 214, while the vertical planes 226, 228 are swept along direction 216. In addition to sweeping in the direction 214, the vertical planes 222, 224 are also swept in the reverse direction. Similarly, in addition to sweeping in the direction 216, the vertical places 226, 228 are also swept in the reverse direction. Such sweeps of the inner moving domains 210 and 212 are referred to bi-frontal sweeps.

The partitioning of the volume 202 depicted in FIG. 2 constitutes a multilevel partitioning, since the partitioning occurs in multiple directions. Note that although FIG. 2 shows the outer sweep of the horizontal planes 206, 208 along just the direction 203, it is noted that the outer sweep can also occur in two opposite directions, including the down direction 203 as well as the opposite up direction.

Although the partitioning example of FIG. 2 shows the outer moving domain 204 being formed based on an outer sweep along the Z axis, it is noted that the outer sweep can be along a different axis, such as the X or Y axis. In such other examples, the inner sweeps for providing the inner moving domains 210, 212 would be sweeps along another direction, such as along the Y or Z axis instead of the X axis.

Based on the partitioning performed according to FIG. 2, three levels of preconditioning can be applied to solve a wave equation for the volume of data 202. A first level preconditioner is applied to the outer moving domains 204 along the Z axis. Solving the wave equation within each outer moving domain 204 is considered a quasi-2D problem, which is solved by the second level preconditioner.

The second level preconditioner is applied by decomposing each outer moving domain 204 in the XY plane. For example, a decomposition of the outer moving domain 204 in the XY plane is shown in FIG. 3. In FIG. 3, a top view of the outer moving domain 204 is shown, where the top view is a view in a direction parallel to the Z axis. The decomposition in FIG. 3 involves splitting the outer moving domain 204 into multiple domains in the XY plane. FIG. 3 shows an example where there are four domains in a Cartesian arrangement, identified as domains (1,1), (1,2), (2,1), and (2,2). In different examples, the decomposition can split the outer moving domain 204 into just two domains, or more than four domains, in the XY plane. Following decomposition in the XY plane, the second level preconditioner stitches the multiple domains together using a stitching technique discussed further below.

A third level preconditioner may be applied to solve the wave equation within each of the quasi-2D domains obtained by partitioning the volume in the XY plane, as shown in FIG. 2. Within each of these domains, the preconditioner uses inner moving domains 210, 212 shown in FIG. 2. Note that the first and third level preconditioners can be sweeping preconditioners, which in some implementations may use overlapping domains.

The first level preconditioner, second level preconditioner, and third level preconditioner together form a multilevel preconditioner. The solving of the wave equation in the respective domains is performed in a recursive fashion, with the multilevel preconditioner iteratively applied as additional domains are produced based on the sweeping along the Z and X axes shown in FIG. 2. As discussed above, these axes may be permuted to other directions.

A seismic wave equation, and more specifically a Helmholtz equation, written in the frequency domain can be represented as follows:

$$ρ {ω}^{2} {u}_{i} + \frac{\partial }{\partial {x}_{j}} ( {c}_{ijkl} \frac{\partial {u}_{k}}{\partial {x}_{i}} ) = {f}_{i} \qquad ( Eq . 1 )$$

where uᵢ, i=1, 2, 3 (corresponding to the X, Y, Z axes, respectively) represents the seismic velocity (or other measured survey data), c represents a material stiffness, p is the mass density, and f is a source term (which represents a survey source).

Although the foregoing refers to using the parallel preconditioner 114 of FIG. 1 to solve a seismic wave equation such as provided above in Eq. 1, it is noted that in alternative implementations, the parallel preconditioner 114 can also be applied to solve wave equations (also referred to as Maxwell equations) for electromagnetic waves.

Iterative solution of the frequency domain wave equation written in abstract form as Au=f can be accelerated by choosing a preconditioner M (preconditioner 114 in FIG. 1) such that M⁻¹ is an approximate inverse of A (which is a matrix based on Eq. 1 above). Solving the wave equation can be performed by solving the system M⁻¹Au=M⁻¹. The preconditioner M can be a multilevel preconditioner, as discussed above.

As noted above, the multilevel preconditioner M can include a first level preconditioner that provides approximate solutions for the outer moving domains 204. As an example, the first level preconditioner can include a preconditioner as discussed in B. Engquist and L. Ying, “SWEEPING PRECONDITIONER FOR THE HELMHOLTZ EQUATION: MOVING PERFECTLY MATCHED LAYERS”, Multiscale Modeling and Simulation, v. 9, No. 2, pp. 686-710 (2011). In other implementations, the first level preconditioner can include a different type of sweeping preconditioner or solver that approximates a solution within a specific domain.

The multilevel preconditioner M further decomposes each outer moving domain 204 in the XY plane into multiple domains, such as depicted in FIG. 3. The domains in the XY direction are then stitched together using an optimized Schwarz method, or other method which stitches domains using an approximate Dirichlet-to-Neumann (DtN) map as discussed further below. In addition, the multilevel preconditioner M may also include a third level preconditioner that includes an iterative solver for the inner moving domains 210 and 212 as shown in FIG. 2.

In some implementations, the outer and inner moving domains for the sweeping preconditioner depicted in FIG. 2 can also be referred to as moving perfectly matched layers (PMLs), and the preconditioners used to solve a wave equation in the respective domains can be referred to as moving PML preconditioners that are applied in a recursive manner. In other implementations, the sweeping preconditioner can use overlapping or non-overlapping domains with a source transfer mechanism between domains.

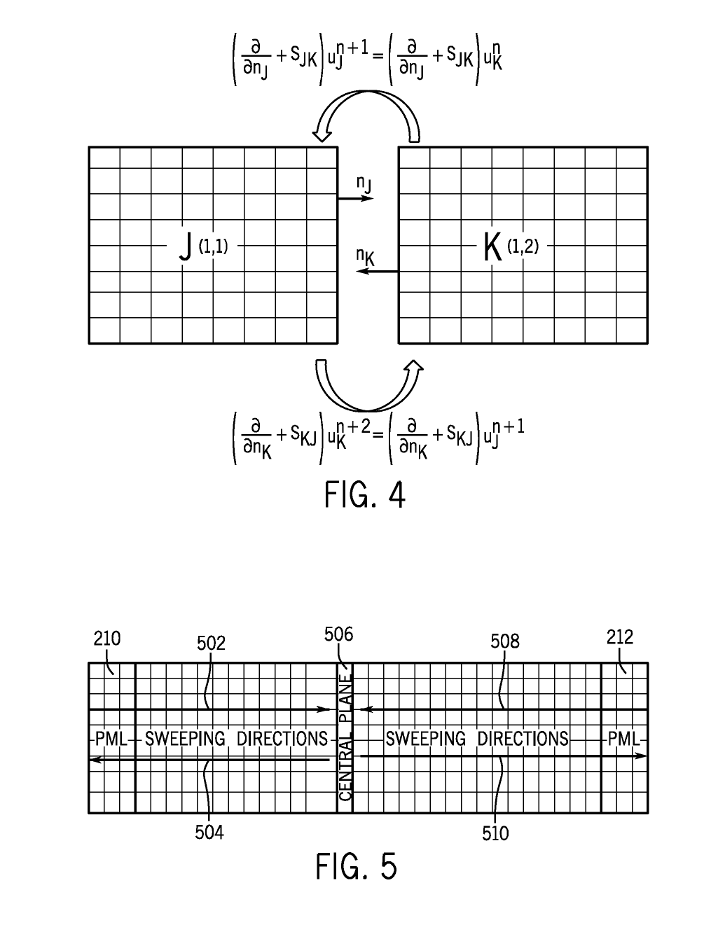

An example of the optimized Schwarz method is depicted in FIG. 4, which shows the stitching of two domains J and K, which can be two of the domains via mixed boundary conditions depicted in FIG. 3. The stitching depicted in FIG. 4 is applied to couple domains in the XY decomposition of FIG. 3.

It is assumed that the preconditioner applied on each domain (i j) in FIGS. 3 and 4 is given by Mᵢⱼ, which accounts for the boundary conditions of the form

$$\frac{\partial u}{\partial n} + Su = 0$$

on the boundary to each domain with outward normal n (n_{J} and n_{K} shown in FIG. 4).

More specifically, the boundary conditions that stitch the domains J and K together are expressed as follows.

$$( \frac{\partial }{\partial {n}_{J}} + {S}_{JK} ) {u}_{J}^{n + 1} = ( \frac{\partial }{\partial {n}_{J}} + {S}_{JK} ) {u}_{K}^{n} and \\ ( \frac{\partial }{\partial {n}_{k}} + {S}_{KJ} ) {u}_{K}^{n + 2} = ( \frac{\partial }{\partial {n}_{k}} + {S}_{KJ} ) {u}_{J}^{n + 1} .$$

In the foregoing, S_{JK} is an operator that maps a wavefield on the interface between domains J and K. The parameter u represents the measured survey data (e.g. pressure or velocity if the survey data is seismic survey data). The parameter u can alternatively represent EM survey data.

The Boundary Conditions

$$( \frac{\partial }{\partial {n}_{J}} + {S}_{JK} ) {u}_{J}^{n + 1} = ( \frac{\partial }{\partial {n}_{J}} + {S}_{JK} ) {u}_{K}^{n} and \\ ( \frac{\partial }{\partial {n}_{k}} + {S}_{KJ} ) {u}_{K}^{n + 2} = ( \frac{\partial }{\partial {n}_{k}} + {S}_{KJ} ) {u}_{J}^{n + 1}$$

are imposed to ensure that pressure (or other wave data) is continuous between domains J and K.

Thus, according to FIG. 4, an approximate solution is provided for a wave equation within each of domains J and K, and these approximate solutions are then stitched together based on the boundary conditions discussed above.

The multilevel preconditioner M coupling the multiple domains can be written in additive Schwarz form as

$${M}^{- 1} = {\sum }_{i , j} {P}_{i , j} {M}_{i , j}^{- 1} {R}_{i , j} ,$$

where Pᵢⱼ and Rᵢⱼ are called prolongation and restriction operators, respectively, for the domain (i,j).

The prolongation operator P is an operator that injects a wavefield on each domain (i, j) back to an entire grid. The entire grid is the plurality of the multiple domains (i, j). The restriction operator R is an operator that restricts the wavefield on each domain (i, j).

In other implementations, other techniques for performing coupling of domains can be employed.

FIG. 5 shows the bi-frontal sweeping within each outer moving domain 204, to produce the inner moving domains 210 and 212. The sweeping directions for the inner moving domain 210 are depicted as 502 and 504. The inner moving domain 210 is swept in direction 502 from the left to the right to a central plane 506. The inner moving domain 210 is then swept backwards in direction 504 back to the left edge. Similarly, the inner moving domain 212 is swept in directions 508 and 510, between a right boundary and the central plane 506.

The bi-frontal sweeping thus starts at the left and right boundaries, and sweeps towards the central plane 506. The bi-frontal sweeping then sweeps backwardly to the left and right boundaries.

The multilevel preconditioner M can be applied to acoustic full waveform inversion with a discretization based on any form of finite-difference, finite element or spectral methods. The multilevel preconditioner can also extend to anisotropic and elastic media.

Additionally, a constrained residual subspace procedure can be used to accelerate preconditioning of the elastic case by projecting the elastic residual onto a pseudo-acoustic (or other lower dimensional) subspace. The constrained residual subspace procedure involves first approximating an elastic wave equation by a related pseudo-acoustic equation. The pseudo-acoustic equation can be used to accelerate convergence in the elastic case, by correcting for errors in the elastic solution using pseudo-acoustic terms of the pseudo-acoustic equation.

The constrained residual subspace procedure can be performed for greater computation efficiency as compared to a procedure that attempts to solve an elastic wave equation directly. It is noted that preconditioning elastic anisotropy using a pseudo-acoustic operator is also possible. This may be useful in cases where a storage subsystem of a system is finite and the system can tolerate some extra iterations. Specifically, a pseudo-acoustic operator Aₚ (a pseudo-acoustic wave equation in the frequency domain) can be derived from the elastic operator Aₑ by Aₚ=RₑₚW₁AₑRₑₚ^{T}, where Rₑₚ is a projection operator and W₁ is a weight. Rₑₚ projects errors in the elastic solution to errors in the pseudo-acoustic solution. For example, Rₑₚ may take elastic sources and define a pseudo-pressure.

A preconditioner for Aₑ is then given by Mₑ⁻¹=Rₑₚ^{T}W₂Mₐ⁻¹Rₑₚ, where Mₐ is a preconditioner (which may be a multilevel preconditioner) for the acoustic system and W₂ is a weight.

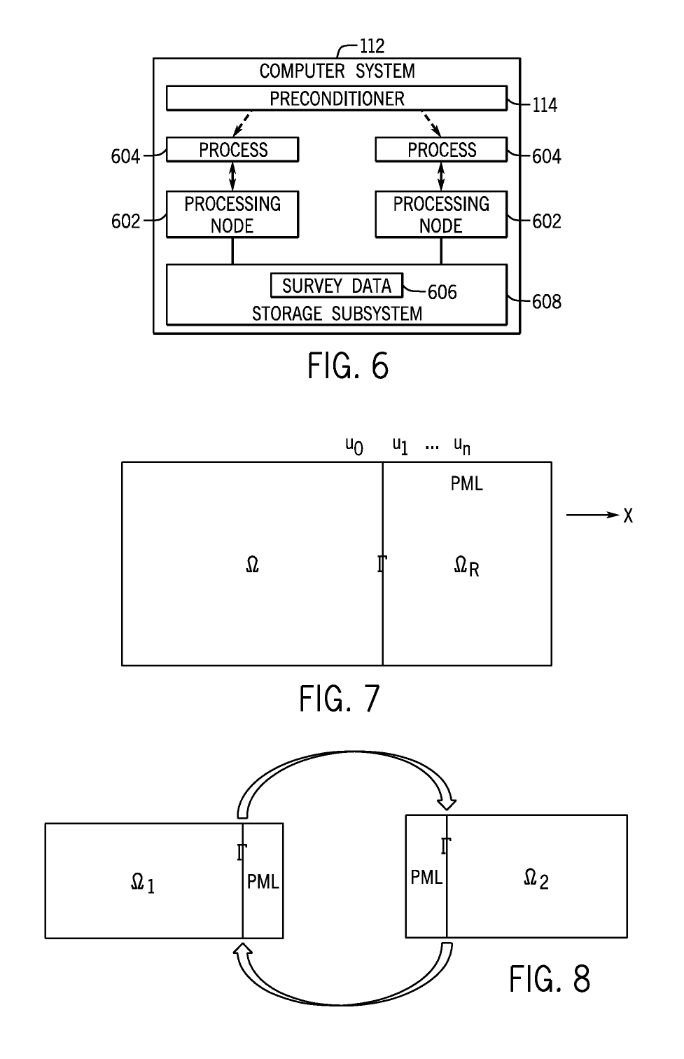

FIG. 6 is a block diagram of an example of the computer system 112 of FIG. 1. The computer system 112 includes multiple processing nodes 602, where each processing node 602 can include a processor, a collection of processors, a computer, or a collection of computers. The tasks of the preconditoner 114 can be executed by multiple processes or threads 604 invoked by the preconditoner 114. Each process or thread 604 includes machine-executable code for performing specified tasks. For example, the processes 604 can perform the tasks of the various different level preconditioners discussed above for respective different domains.

In some implementations, the processes 604 can communicate with each other through message passing. By passing messages between the processes 604, these processes 604 are able to couple solutions obtained by the respective processes 604. In other implementations, other techniques for communications between the processes 604 can be employed.

The computer system 112 further includes a storage subsystem 608 for storing survey data 606. The survey data 606 is retrievable by the processes 604 for applying their respective operations.

The different level preconditioners discussed above are iterative solvers that iterate through the various domains. The iterative execution of the solvers can continue until convergence is reached in the results of the solvers. Various convergence criteria can be used to ensure the iterative solution is accurate.

As discussed above, domains in the XY direction (domains in FIG. 3, for example) can be stitched together using an optimized Schwarz method, which provides for an operator S_{JK} that maps a wavefield on the interface between two different domains, such as domains J and K in FIG. 4. More simply, the operator S_{JK} can simply be referred to as an operator S, which is defined on the interfaces across adjacent domains. The operator S is an approximation of a Dirichlet-to-Neumann (DtN) map at domain boundaries. Since calculating a Dirichlet-to-Neumann map may be impractical from a computation perspective, an approximation is derived by computing the operators S for use with the multilevel preconditioner. The accuracy of the operator S is determined by the numerical approximation of the DtN operator across domain boundaries. In some implementations, a perfectly matched layer (PML) can be used to construct an approximate DtN operator. In other implementations, a perfectly matched discrete layer method (PMDL) method can be used to approximate a DtN operator.

To construct the approximate DtN operator on each face of a domain Ω, such as the domain depicted in FIG. 7, in one implementation, an auxiliary perfectly matched layer (PML) is added to an interface Γ of the domain Ω with coefficients determined by an optimization procedure.

For ease of explanation, a one-dimensional (1D) acoustic example with a PML Ω_{R} is added only to the right interface in the X axis, with a simple three-point finite difference discretization shown in FIG. 7. The discretization in the combined domain Ω and Ω_{R} is as follows:

$$\frac{{u}_{i + 1} - 2 {u}_{i} + {u}_{i + 1}}{{h}^{2}} + {k}^{2} {u}_{i} = {f}_{i}$$

in the interior of Ω, and

$$\frac{1}{{h}_{j}} ( \frac{{u}_{i + 1} - {u}_{i}}{{h}_{i}} - \frac{{u}_{i} - {u}_{i + 1}}{{h}_{i - 1}} ) + {k}^{2} {u}_{i} = 0$$

in Ω and on the interface Γ between Ω and Ω_{R}. The parameter h is the grid size in the interior of Ω, and the optimal grid steps hᵢ and ĥᵢ are complex numbers obtained by an L_{∞} problem in rational interpolation to reduce the numerical reflection on the interface Γ.

The steps hᵢ and ĥᵢ may be used to define the approximate DtN operators used as boundary conditions in an optimized Schwarz method. The DtN operators may be tuned by adjusting the approximation separately for propagating and also for evanescent waves, and over a given range of wavenumbers.

The operator S can be derived by using a complex rational approximation problem to minimize the numerical reflection coefficient for a PML. In some implementations, complex rational approximation may be achieved using Zolotarev polynomials, such as described in Sergei Asvadurov et al., “On Optimal Finite-Difference Approximation of PML,” SIAM Journal on Numerical Analysis, pp. 287-305 (2003), for example. Moreover, complex shifts in the wave number can be used to accelerate convergence. In the acoustic case, this replaces the real wave number k by a new complex wavenumber \sqrt{k²+iε} where ε is an attenuation parameter. The coefficients of the operators S are designed to match the dispersion relation for the discretization in each domain.

DtN operators (using a complex wavenumber) based on complex rational approximation can be used to accelerate domain decomposition used as the engine for frequency domain FWI. The DtN operator is used to connect domains in an optimized Schwarz method. The Schwarz preconditioner is then combined with sweeping or multilevel preconditioning for the frequency domain wave equation, and the combined multilevel preconditioner is used for frequency domain seismic (acoustic or elastic) or EM inversion.

FIG. 8 illustrates the addition of an auxiliary PML on the interface F of each of domains Ω₁ and Ω₂. The boundary conditions for coupling the domains can be similar to the boundary conditions discussed above in connection with FIG. 4.

The multilevel preconditioner according to some implementations can also be used as a fine or coarse grid preconditioner in multiple coarse grids for solving a wave equation in the frequency domain.

Machine-readable instructions of modules described above (including those depicted in FIGS. 1 and 6) are loaded for execution on a processor. A processor can include a microprocessor, microcontroller, processor module or subsystem, programmable integrated circuit, programmable gate array, graphics processing unit, or another control or computing device.

Data and instructions are stored in respective storage devices, which are implemented as one or multiple computer-readable or machine-readable storage media. The storage media include different forms of memory including semiconductor memory devices such as dynamic or static random access memories (DRAMs or SRAMs), erasable and programmable read-only memories (EPROMs), electrically erasable and programmable read-only memories (EEPROMs) and flash memories; magnetic disks such as fixed, floppy and removable disks; other magnetic media including tape; optical media such as compact disks (CDs) or digital video disks (DVDs); or other types of storage devices. Note that the instructions discussed above can be provided on one computer-readable or machine-readable storage medium, or alternatively, can be provided on multiple computer-readable or machine-readable storage media distributed in a large system having possibly plural nodes. Such computer-readable or machine-readable storage medium or media is (are) considered to be part of an article (or article of manufacture). An article or article of manufacture can refer to any manufactured single component or multiple components. The storage medium or media can be located either in the machine running the machine-readable instructions, or located at a remote site from which machine-readable instructions can be downloaded over a network for execution.

In the foregoing description, numerous details are set forth to provide an understanding of the subject disclosed herein. However, implementations may be practiced without some of these details. Other implementations may include modifications and variations from the details discussed above. It is intended that the appended claims cover such modifications and variations.

What is claimed is:

1. A method comprising:
partitioning, by a system including a processor, a volume of data into a plurality of domains;
applying, by the system, a preconditioner in the plurality of domains to provide iterative approximate solutions for the domains; and
stitching, by the system, the approximate solutions for the domains together to provide a solution for a wave equation.

2. The method of claim 1, wherein providing the solution for the wave equation comprises providing the solution for the wave equation in a frequency domain, the method further comprising performing full waveform inversion on the data, wherein the partitioning, the applying, and the stitching are performed as part of the full waveform inversion.

3. The method of claim 1, wherein partitioning the volume into the plurality of domains comprises partitioning the volume in a plurality of directions, and wherein applying the preconditioner comprises applying the preconditioner at a plurality of levels that correspond to the partitioning in the plurality of directions.

4. The method of claim 3, wherein partitioning the volume in the plurality of directions comprises:
partitioning the volume into first domains along a first direction; and
partitioning a particular one of the first domains into multiple second domains along a second direction.

5. The method of claim 4, wherein partitioning the particular first domain into the multiple second domains comprises sweeping in the second direction in the particular first domain to define the multiple second domains.

6. The method of claim 5, wherein the sweeping comprises a recursive bi-directional sweep in opposite directions.

7. The method of claim 1, wherein stitching the approximate solutions for the domains together comprises imposing boundary conditions between adjacent ones of the domains that specify continuity of boundary conditions derived from the data between the successive domains.

8. The method of claim 7, wherein the stitching constructs an operator for an interface between the adjacent domains.

9. The method of claim 8, wherein constructing the operator comprises constructing a Dirichlet-to-Neumann operator.

10. The method of claim 9, wherein constructing the operator comprises adding an auxiliary perfectly matched layer to a face of a respective one of the domains.

11. The method of claim 8, wherein constructing the operator reduces a numerical reflection on the interface.

12. The method of claim 1, further comprising:
using the full waveform inversion to determine properties of a subsurface structure or to generate an image of the subsurface structure.

13. The method of claim 12, wherein the volume of data comprises seismic data from a seismic survey or medical data from a medical scan.

14. A system comprising:
at least one storage medium to store a volume of data derived from measurement data acquired by surveying a target structure; and
at least one processor configured to:
apply a preconditioner across a plurality of domains generated by sweeping across the volume of data, to provide approximate solutions for the respective domains;
stitching the approximate solutions for the domains together to provide a solution for a wave equation.

15. The system of claim 14, wherein the at least one processor comprises a plurality of processors, and applying the preconditioner across the plurality of domains is performed in parallel across the plurality of processors.

16. The system of claim 14, wherein providing the solution for the wave equation is part of applying full waveform inversion on the measurement data to produce an image or model of the target structure.

17. The system of claim 14, wherein the measurement data is selected from the group consisting of seismic survey data and electromagnetic survey data.

18. The system of claim 14, wherein the at least one processor is configured to further approximate an elastic wave equation by a pseudo-acoustic equation, wherein providing the solution for the wave equation comprises providing a solution for the pseudo-acoustic equation.

19. An article comprising at least one non-transitory machine-readable storage medium storing instructions executable by a computer system to:
partition a volume of data into a plurality of domains;
apply a preconditioner in the plurality of domains to provide iterative approximate solutions for the domains; and
stitch the approximate solutions for the domains together to provide a solution for a wave equation

20. The article of claim 19, wherein the partitioning comprises sweeping the volume of data along first and second directions.

21. The article of claim 19, wherein applying the preconditioner comprises performing tasks of the preconditioner using respective processes executable on respective processing nodes of the computer system.

22. The article of claim 19, wherein the stitching is based on boundary conditions defined for respective domains.
